(19) United States
(12) Patent Application Publication
(10) Pub. No.: US 2024/0285565 A1
(43) Pub. Date: Aug. 29, 2024

(54) USE OF (S)-3-AMINO-4-(DIFLUOROMETHYLENYL)CYCLOPENT-1-ENE-1-CARBOXYLIC ACID IN THE TREATMENT OF DIABETES AND PRE-DIABETES

(71) Applicant: Ovid Therapeutics Inc., New York, NY (US)

(72) Inventor: Patrick Sarmiere, New York, NY (US)

(21) Appl. No.: 18/443,583

(22) Filed: Feb. 16, 2024

Related U.S. Application Data
(60) Provisional application No. 63/485,624, filed on Feb. 17, 2023.

(51) Int. Cl. A61K 31/196; A61K 45/06

(52) U.S. Cl. CPC A61K 31/196; A61K 45/06

(57) ABSTRACT

Methods and compositions for treating diabetes or prediabetes include administering (S)-3-amino-4-(difluoromethylenyl)cyclopent-1-ene-1-carboxylic acid or a pharmaceutically acceptable salt thereof to a subject diagnosed with diabetes or prediabetes. In embodiments, administering (S)-3-amino-4-(difluoromethylenyl)cyclopent-1-ene-1-carboxylic acid or a pharmaceutically acceptable salt thereof to a subject diagnosed with diabetes or prediabetes is effective to lower one or more of HbA1c level, fasting plasma glucose level, 2-hour oral glucose tolerance test (OGTT) result level, and random plasma glucose level. (S)-3-amino-4-(difluoromethylenyl)cyclopent-1-ene-1-carboxylic acid or a pharmaceutically acceptable salt thereof may optionally be administered in combination with a hypoglycemic agent.

### CROSS REFERENCE TO RELATED APPLICATION

This application claims benefit of and priority to U.S. Provisional Application No. 63/485,624, filed on Feb. 17, 2023, and which is incorporated herein by reference in its entirety.

### TECHNICAL FIELD

Treatment of diabetes and prediabetes with (S)-3-amino-4-(difluoromethylenyl)cyclopent-1-ene-1-carboxylic acid or pharmaceutically acceptable salt thereof.

### BACKGROUND

According to the World Health Organization, diabetes is a chronic disease that occurs either when the pancreas does not produce enough insulin or when the body cannot effectively use the insulin it produces. Insulin regulates (decreases) the concentration of blood sugar (glucose). It is a peptide hormone produced by beta cells of the pancreatic islets. Insulin regulates the metabolism of carbohydrates, fats and protein by promoting the absorption of glucose from the blood into fat, liver and skeletal muscle cells. Pancreatic beta cells (β cells) are sensitive to glucose concentrations in the blood. In non-diabetics, when glucose concentrations in the blood are high, the pancreatic beta cells secrete insulin into the blood; when glucose levels are low, secretion of insulin is inhibited. Pancreatic alpha cells secrete glucagon, another peptide hormone, into the blood to raise the concentration of glucose in the blood in the opposite manner, i.e., increased secretion when blood glucose is low, and decreased secretion when glucose concentrations are high. The secretion of insulin and glucagon into the blood in response to the blood glucose concentration is the primary mechanism responsible for keeping the glucose levels in the extracellular fluids within narrow limits.

Hyperglycemia, or high blood sugar, is a common effect of uncontrolled diabetes and over time can lead to serious damage to many of the body's systems, especially the nerves and blood vessels. According to the World Health Organization the number of people with diabetes has risen from 108 million in 1980 to 422 million in 2014. In 2014, 8.5% of adults aged 18 years and older had diabetes. In 2012 diabetes was the direct cause of 1.5 million deaths and high blood glucose was the cause of another 2.2 million deaths. Diabetes is a major cause of blindness, kidney failure, heart attacks, stroke and lower limb amputation. Adults with diabetes have a 2-3-fold increased risk of heart attacks and strokes. Combined with reduced blood flow, neuropathy (nerve damage) in the feet increases the chance of foot ulcers, infection and eventual need for limb amputation. Diabetes is among the leading causes of kidney failure.

Type 1 diabetes (previously known as insulin-dependent, juvenile or childhood-onset) is characterized by deficient insulin production and typically requires daily administration of insulin. The majority of cases of Type 1 diabetes are primarily due to pancreatic islet beta-cell destruction. Type 1 diabetics are prone to ketoacidosis. Symptoms of type 1 diabetes include excessive excretion of urine (polyuria), thirst (polydipsia), constant hunger, weight loss, vision changes and fatigue. These symptoms may occur suddenly. Type 2 diabetes (formerly called non-insulin-dependent or adult-onset) results from the body's ineffective use of insulin. The majority of people with diabetes around the world have type 2 diabetes. Type 2 diabetes has been correlated to excess body weight and physical inactivity. Symptoms may be similar to those of Type 1 diabetes, but are often less marked. Sleep apnea has been correlated to an increased risk of developing Type 2 diabetes. See, Botros et al., Amer. J. Med., 122(12), 1122-1127 (2009). Over 50% of Type 2 diabetics have sleep apnea. Until recently, Type 2 diabetes was seen only in adults but it is now also occurring increasingly frequently in children. In type 1 diabetes, 70% of pancreatic β-cells are destroyed while in type 2 diabetes 40-60% of pancreatic β-cells are destroyed at the time of diagnosis. Al-Kuraishy et al., Int J Prev Med. 2021 Feb. 24; 12:19.

Impaired glucose tolerance (IGT) and impaired fasting glycemia (IFG) (collectively referred to as “pre-diabetes” herein) are intermediate conditions in the transition between normality and diabetes. IFG and IGT are not interchangeable and represent different abnormalities of glucose regulation, one in the fasting state and one post-prandial. People with IGT or IFG are at high risk of progressing to type 2 diabetes, although it is not inevitable.

According to the guidelines of the American Diabetes Association criteria for diabetes diagnosis involves four options: a fasting plasma glucose level greater than or equal to 126 mg/dl, a 2-hour oral glucose tolerance test (OGTT) providing a plasma glucose value of greater than or equal to 200 mg/dl, an HbA1c value greater than or equal to 6.5%, or a random plasma glucose level greater than or equal to 200 mg/dl in individuals with symptoms of hyperglycemia or hyperglycemic crisis. Pre-diabetes is defined as having a fasting glucose level of between 100 mg/dl and 125 mg/dl, a 2-hour OGTT plasma glucose level of between 140 mg/dl and 199 mg/dl, or an HbA1c value between 5.7 and 6.4%. Pre-diabetes may be considered to be a major risk factor for the development of type 2 diabetes mellitus, cardiovascular disease and mortality.

In the monitoring of the treatment of diabetes, the HbA1c value, which is the product of a non-enzymatic glycation of the hemoglobin B chain, may be considered to be an important parameter. HbA1c values depend on the blood sugar level and the life time of erythrocytes in the blood. HbA1c values typically reflect the average blood sugar level 4-12 weeks prior to removal and testing of a patient's blood. Diabetic patients whose HbA1c level has been well controlled over a long time treatment (i.e. <6.5% of the total hemoglobin in the sample) are typically better protected from diabetic microangiopathy. The available treatments for diabetes can give the diabetic an average improvement in their HbA1c level of the order of 1.0-1.5%. However, this reduction in the HbA1C level may not be sufficient in all diabetics to bring them into the desired target range of <7.0%, preferably <6.5% and more preferably <6% HbA1c.

Within glycemic control, in addition to improvement of the HbA1c level, other recommended therapeutic goals for type 2 diabetes patients are improvement of fasting plasma glucose (FPG) and of postprandial plasma glucose (PPG) levels to normal or as near normal as possible. Desired target ranges of preprandial (fasting) plasma glucose can be, e.g., 90-130 mg/dL or <110 mg/dL, and of two-hour postprandial plasma glucose can be, e.g., <180 mg/dL or <140 mg/dL.

Diet therapy and exercise therapy are frequently considered to be essential in the treatment of diabetes. When these therapies do not sufficiently control the condition of patients (especially their blood sugar levels), an oral or non-oral antidiabetic agent can be used for the treatment of diabetes. Conventional antidiabetic or hypoglycemic agents include, without limitation, biguanides, dipeptidyl peptidase-4 (DPP-4) inhibitors, sulphonylureas, thiazolidinediones, meglitinides (aka_glinides), alpha-glucosidase blockers, GLP-1 and GLP-1 analogs, as well as insulin and insulin analogs.

Although intensive treatment of hyperglycemia can reduce the incidence of chronic damage, many patients with diabetes remain inadequately treated, partly because of limitations in long term efficacy, tolerability and dosing inconvenience of existing antihyperglycemic therapies. The use of conventional hypoglycemic agents can be associated with various adverse effects. For example, metformin can be associated with lactic acidosis or gastrointestinal side effects; sulfonylureas, meglitinides (glinides) and insulin or insulin analogues can be associated with hypoglycemia or weight gain; thiazolidinediones can be associated with edema, bone fracture, weight gain or heart failure/cardiac effects; and alpha-glucosidase blockers and GLP-1 or GLP-1 analogues can be associated with gastrointestinal adverse effects (e.g. dyspepsia, flatulence or diarrhea, or nausea or vomiting). There exists a continuing need for improved antihyperglycemic therapy in the treatment of metabolic diseases.

Vigabatrin is a gamma aminobutyric acid aminotransferase (GABA-AT) inhibitor that has been used to treat treatment resistant epilepsy and infantile spasms. Vigabatrin has been associated with some potentially serious side effects. Its use has been limited due to potential retinal toxicity and subsequent visual field defects. (1S,3S)-3-amino+difluoromethylenyl-1-cyclopentanoic acid (also known as CPP-115) is a GABA-AT inhibitor which is 186 times more efficient in inactivating GABA-AT than vigabatrin. Preclinical data for CPP-115 has been reported to show that significantly lower drug dosages afford comparable pharmacokinetics, improved tolerability, and a more favorable toxicity profile when compared with vigabatrin. See, Prescot et al., Neuropsychopharmacology (2018) 43, 646-654. See also, U.S. Pat. No. 9,993,449, incorporated herein by reference. (S)-3-amino-4-(difluoromethylenyl)cyclopent-1-ene-1-carboxylic acid (also known as OV329) is a GABA-AT inhibitor that has been shown to be 9.8 times more efficient as an inactivator of GABA-AT than CPP-115. Id.

### SUMMARY

Methods and compositions for treating diabetes and prediabetes are provided. In embodiments, methods for treating diabetes or prediabetes include administering an effective amount of (S)-3-amino-4-(difluoromethylenyl)cyclopent-1-ene-1-carboxylic acid or a pharmaceutically acceptable salt thereof to a subject in need thereof. In embodiments, compositions for treating diabetes or prediabetes which include (S)-3-amino-4-(difluoromethylenyl)cyclopent-1-ene-1-carboxylic acid or a pharmaceutically acceptable salt thereof are administered to a subject in need thereof. In embodiments, methods of treating diabetes or prediabetes include administering (S)-3-amino-4-(difluoromethylenyl)cyclopent-1-ene-1-carboxylic acid or a pharmaceutically acceptable salt thereof to a subject in need thereof to provide improvement in one or more symptoms of diabetes or prediabetes in the subject. In embodiments, methods of treating diabetes or prediabetes include administering (S)-3-amino-4-(difluoromethylenyl)cyclopent-1-ene-1-carboxylic acid or a pharmaceutically acceptable salt thereof to a subject in need thereof to provide improvement in symptoms of diabetes or prediabetes in the subject the next day after administering (S)-3-amino-4-(difluoromethylenyl)cyclopent-1-ene-1-carboxylic acid or a pharmaceutically acceptable salt thereof. In embodiments, an effective amount of (S)-3-amino-4-(difluoromethylenyl)cyclopent-1-ene-1-carboxylic acid or a pharmaceutically acceptable salt thereof is between about 0.01 mg to about 750 mg. In embodiments, an effective amount of (S)-3-amino-4-(difluoromethylenyl)cyclopent-1-ene-1-carboxylic acid or a pharmaceutically acceptable salt thereof is administered to a subject once, twice, three times or four times daily.

In embodiments, methods of treating diabetes or prediabetes include administering (S)-3-amino-4-(difluoromethylenyl)cyclopent-1-ene-1-carboxylic acid or a pharmaceutically acceptable salt thereof to a subject in need thereof to lower one or more of HbA1c level, fasting plasma glucose level, 2-hour oral glucose tolerance test (OGTT) result level, and random plasma glucose level. In embodiments, the diabetes is type 2 diabetes. In embodiments, the diabetes is type 1 diabetes. In embodiments, the HbA1c level is lowered by an amount greater than 0.25%. In embodiments, the HbA1c level is lowered by an amount greater than 0.5%. In embodiments, the HbA1c level is lowered by an amount greater than 0.75%. In embodiments, the HbA1c level is lowered by an amount greater than 1.0%. In embodiments, the HbA1c level is lowered by an amount greater than 1.5%. In embodiments, (S)-3-amino-4-(difluoromethylenyl)cyclopent-1-ene-1-carboxylic acid or a pharmaceutically acceptable salt thereof is administered in combination with one or more hypoglycemic agents such as a biguanide, a dipeptidyl peptidase-4 (DPP-4) inhibitor, a sulphonylurea, a thiazolidinedione, a meglitinide (glinide), an alpha-glucosidase blocker, an glucagon-like peptide-1 receptor agonist, insulin and an insulin analog. In embodiments, the combination of (S)-3-amino-4-(difluoromethylenyl)cyclopent-1-ene-1-carboxylic acid or a pharmaceutically acceptable salt thereof and the one or more hypoglycemic agents provides a therapeutic benefit greater than the additive effect of administering the same dosage of each of (S)-3-amino-4-(difluoromethylenyl)cyclopent-1-ene-1-carboxylic acid or a pharmaceutically acceptable salt thereof and the hypoglycemic agents alone.

In embodiments, methods of treating diabetes and sleep apnea include administering (S)-3-amino-4-(difluoromethylenyl)cyclopent-1-ene-1-carboxylic acid or a pharmaceutically acceptable salt thereof to a subject diagnosed with the diabetes and sleep apnea in an amount effective to reduce episodes of sleep apnea.

### DETAILED DESCRIPTION

Provided herein are methods and compositions for use in treating diabetes, including type 1 diabetes, type 2 diabetes, and pre-diabetes. In embodiments, administration of an effective amount of (S)-3-amino-4-(difluoromethylenyl)cyclopent-1-ene-1-carboxylic acid or a pharmaceutically acceptable salt thereof, alone or optionally in combination with one or more hypoglycemic agents such as a biguanide, a dipeptidyl peptidase-4 (DPP-4) inhibitor, a sulphonylurea, a thiazolidinedione, a meglitinide (glinide), an alpha-glucosidase blocker, an glucagon-like peptide-1 receptor agonist, insulin or an insulin analog can reduce symptoms of, prevent, slow the progression of, or delay a metabolic disorder such as, e.g., type 1 diabetes, type 2 diabetes, pre-diabetes, impaired glucose tolerance (IGT), impaired fasting blood glucose (IFG), hyperglycemia, and/or postprandial hyperglycemia.

Methods and compositions involving an effective amount of (S)-3-amino-4-(difluoromethylenyl)cyclopent-1-ene-1-carboxylic acid or a pharmaceutically acceptable salt thereof for use in treating diabetes, including type 1 diabetes, type 2 diabetes, and pre-diabetes disclosed herein are used for improving glycemic control. “Improvement of glycemic control”, “improving glycemic control” or “glycemic control” refers to improvement of glucose tolerance, improvement of postprandial plasma glucose concentration, improvement of fasting plasma glucose concentration, improvement of the HbA1c value or/and improvement of fasting plasma insulin concentration.

Methods and compositions involving an effective amount of (S)-3-amino-4-(difluoromethylenyl)cyclopent-1-ene-1-carboxylic acid or a pharmaceutically acceptable salt thereof for use in treating diabetes or prediabetes disclosed herein improve, reduce or alleviate symptoms or conditions associated with diabetes or prediabetes. Conditions associated with diabetes or prediabetes can include, e.g., sleep apnea, obesity, dyslipidemia, hyperlipidemia, hypercholesterolemia, hypertension, atherosclerosis, endothelial dysfunction, osteoporosis, chronic systemic inflammation, non-alcoholic fatty liver disease (NAFLD), retinopathy, neuropathy, nephropathy and/or metabolic syndrome. Methods and compositions involving an effective amount of (S)-3-amino-4-(difluoromethylenyl)cyclopent-1-ene-1-carboxylic acid or a pharmaceutically acceptable salt thereof for use in treating diabetes or prediabetes disclosed herein can improve glycemic control, e.g., reduce fasting plasma glucose, reduce postprandial plasma glucose and/or reduce HbA1c. Methods and compositions involving an effective amount of (S)-3-amino-4-(difluoromethylenyl)cyclopent-1-ene-1-carboxylic acid or a pharmaceutically acceptable salt thereof for use in treating diabetes or prediabetes disclosed herein can prevent, slow, delay or reverse progression from impaired glucose tolerance (IGT), impaired fasting blood glucose (IFG), insulin resistance from metabolic syndrome, to type 2 diabetes.

Methods and compositions involving an effective amount of (S)-3-amino-4-(difluoromethylenyl)cyclopent-1-ene-1-carboxylic acid or a pharmaceutically acceptable salt thereof for use in treating diabetes or prediabetes disclosed herein can prevent, reduce the risk, slow the progression, delay or treat complications of diabetes or prediabetes such as micro- and macrovascular diseases including nephropathy, micro- or macroalbuminuria, proteinuria, retinopathy, cataracts, neuropathy, learning or memory impairment, neurodegenerative or cognitive disorders, cardio- or cerebrovascular diseases, tissue ischemia, diabetic foot ulcers, atherosclerosis, hypertension, endothelial dysfunction, myocardial infarction, acute coronary syndrome, unstable angina pectoris, stable angina pectoris, peripheral arterial occlusive disease, cardiomyopathy, heart failure, heart rhythm disorders, vascular restenosis, and/or stroke. Methods and compositions involving an effective amount of (S)-3-amino-4-(difluoromethylenyl)cyclopent-1-ene-1-carboxylic acid or a pharmaceutically acceptable salt thereof for use in treating diabetes or prediabetes disclosed herein can prevent, slow the progression of, delay or treat type 2 diabetes with primary or secondary failure to conventional (oral) hypoglycemic mono- or combination therapy. Methods and compositions involving an effective amount of (S)-3-amino-4-(difluoromethylenyl)cyclopent-1-ene-1-carboxylic acid or a pharmaceutically acceptable salt thereof for use in treating diabetes or prediabetes disclosed herein can achieve a reduction in the dose of conventional hypoglycemic medication required for adequate therapeutic effect, thereby reducing the risk for adverse effects associated with conventional hypoglycemic medication. Methods and compositions involving an effective amount of (S)-3-amino-4-(difluoromethylenyl)cyclopent-1-ene-1-carboxylic acid or a pharmaceutically acceptable salt thereof for use in treating diabetes or prediabetes disclosed herein can maintain and/or improve insulin sensitivity and/or treat or prevent hyperinsulinemia and/or insulin resistance.

In embodiments, methods and compositions involving an effective amount of (S)-3-amino-4-(difluoromethylenyl)cyclopent-1-ene-1-carboxylic acid or a pharmaceutically acceptable salt thereof for use in treating diabetes or prediabetes disclosed herein are used to treat inadequate or insufficient glycemic control in a subject with diabetes or prediabetes. Inadequate or insufficient glycemic control may be considered to be a condition wherein patients exhibit HbA1c values above 6.0%, e.g., above 6.5%, above 7.0%, above 7.5%, above 8%, above 8.5%, above 9%, above 9.5%, above 10%, above 10.5%, above 11%, or any value between 6.0% and 11.0%. For example, patients with inadequate or insufficient glycemic control may include patients having a HbA1c value from 6.5 to 7.0%, 7.0-7.5%, 7.5 to 10% or from 7.5 to 11%. For example, inadequately controlled patients can refer to patients with poor glycemic control including, without being limited, patients having a HbA1c value ≥9%.

In embodiments, methods and compositions involving an effective amount of (S)-3-amino-4-(difluoromethylenyl)cyclopent-1-ene-1-carboxylic acid or a pharmaceutically acceptable salt thereof for use in treating diabetes or prediabetes disclosed herein lower HbA1c levels by an amount greater than 0.25%. In embodiments, methods and compositions involving an effective amount of (S)-3-amino-4-(difluoromethylenyl)cyclopent-1-ene-1-carboxylic acid or a pharmaceutically acceptable salt thereof for use in treating diabetes or prediabetes disclosed herein lower HbA1c levels by an amount greater than 0.5%. In embodiments, methods and compositions involving an effective amount of (S)-3-amino-4-(difluoromethylenyl)cyclopent-1-ene-1-carboxylic acid or a pharmaceutically acceptable salt thereof for use in treating diabetes or prediabetes disclosed herein lower HbA1c levels by an amount greater than 0.75%. In embodiments, methods and compositions involving an effective amount of (S)-3-amino-4-(difluoromethylenyl)cyclopent-1-ene-1-carboxylic acid or a pharmaceutically acceptable salt thereof for use in treating diabetes or prediabetes disclosed herein lower HbA1c levels by an amount greater than 1.0%. In embodiments, methods and compositions involving an effective amount of (S)-3-amino-4-(difluoromethylenyl)cyclopent-1-ene-1-carboxylic acid or a pharmaceutically acceptable salt thereof for use in treating diabetes or prediabetes disclosed herein lower HbA1c levels by an amount greater than 1.25%. In embodiments, methods and compositions involving an effective amount of (S)-3-amino-4-(difluoromethylenyl)cyclopent-1-ene-1-carboxylic acid or a pharmaceutically acceptable salt thereof for use in treating diabetes or prediabetes disclosed herein lower HbA1c levels by an amount greater than 1.5%.

The structure of (S)-3-amino-4-(difluoromethylenyl)cyclopent-1-ene-1-carboxylic acid may be represented as follows:

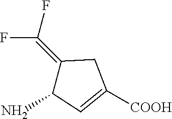 [SMILES: N[C@H]1C=C(C(=O)O)CC1=C(F)F]

In embodiments, (S)-3-amino-4-(difluoromethylenyl)cyclopent-1-ene-1-carboxylic acid may be provided as an acid addition salt, a zwitter ion hydrate, zwitter ion anhydrate, hydrochloride or hydrobromide salt, or in the form of the zwitter ion monohydrate. Acid addition salts, include but are not limited to, maleic, fumaric, benzoic, ascorbic, succinic, oxalic, bis-methylenesalicylic, methanesulfonic, ethane-disulfonic, acetic, propionic, tartaric, salicylic, citric, gluconic, lactic, malic, mandelic, cinnamic, citraconic, aspartic, stearic, palmitic, itaconic, glycolic, pantothenic, p-amino-benzoic, glutamic, benzene sulfonic or theophylline acetic acid addition salts, as well as the 8-halotheophyllines, for example 8-bromo-theophylline. In embodiments, inorganic acid addition salts, including but not limited to, hydrochloric, hydrobromic, hydroiodic, sulfuric, sulfamic, phosphoric or nitric acid addition salts may be used.

The pathogenesis of diabetes and prediabetes can begin with an inflammatory cascade. Autoimmunity in diabetes and prediabetes can start at the molecular and cellular level. For example, serum cytokine levels can increase. Some patients eventually transition from autoimmunity to immune-mediated inflammation primarily focused in the pancreas. The pancreas is infiltrated by immune cells which include innate immune cells (monocytes, dendritic cells, mast cells) and adaptive immune cells (T-helper 1, Th1); T-helper 17, Th17), B cells, and plasma cells. Neutrophils may not ordinarily be present in the pancreas but egress from the blood to the pancreas. Cytokines and chemokines such as tumor necrosis factor (TNF), interleukin-6 (IL-6), and granulocyte-monocyte colony-stimulating factor (GM-CSF) can activate endothelial cells and attract immune cells within the pancreas.

Gamma-amino butyric acid (GABA) is the chief inhibitory neurotransmitter in the mature mammalian CNS. Outside the brain, GABA is produced by pancreatic β-cells as well as T cells and macrophages that express all components of GABAergic system, including its receptors, transporters, and metabolic enzymes. GABA acts as a negative regulator of macrophage production of inflammatory cytokines and T cell activation by blocking calcium signaling and NFκB activity.

Many different types of murine and human immune cells express various GABA receptors (GABA-Rs). See, Tian et al., Sci Rep 11, 5402 (2021). https://doi.org/10.1038/s41598-021-84751-3. GABA has been shown to limit murine T cell production of IL-21, IFNγ2-4, TNFα3, and IL-123 while promoting TGFß and Treg responses. Id. Antigen presenting cells (APCs) such as macrophages also express GABA_{A}-Rs and their activation inhibits their inflammatory activity. Id. For example, GABA or a GABA_{A}-R agonist reduced the secretion of IL-6, IL-1ß, IL-12 and/or TNFα from LPS-stimulated murine macrophages. Id.

Pancreatic β-cells synthesize GABA from glutamic acid by glutamic acid decarboxylase (GAD). Sec, Al-Kuraishy et al., Int J Prev Med. 2021 Feb. 24; 12:19. The potential role of GABA in pancreatic islets is regulation of hormone secretions, inhibition of immune response, improved β-cells survival, and changing of α cells into β-cells. Id. Notably, auto-antibodies against GAD are linked with the development of type 1 diabetes in which β-cell mass is reduced and eventually disappears. Id. Glucagon-like peptide (GLP-1) activates pancreatic GAD and leads to elevation of β-cell GABA concentrations which increase insulin synthesis and secretions. Id. GABA through both of its receptors preserves β-cells mass and prevents the progression of type 1 diabetes and type 2 diabetes due to immune-regulatory and anti-inflammatory effects. Id. GABA exerts a trophic effect on β-cells through activation of PI3K/Akt pathway. Id. GABA content in beta cells is depleted and secretion is disrupted in islets from patients with type 1 and type 2 diabetes, suggesting that loss of GABA as a synchronizing signal for hormone output may correlate with diabetes pathogenesis. Menegaz et al., Nat Metab 1, 1110-1126 (2019) Abstract.

(S)-3-amino-4-(difluoromethylenyl)cyclopent-1-ene-1-carboxylic acid is a gamma-aminobutyric acid aminotransferase (GABA-AT) inhibitor. GABA-AT is a pyridoxal 5′-phosphate-dependent enzyme responsible for the degradation of the inhibitory neurotransmitter GABA. (S)-3-amino-4-(difluoromethylenyl)cyclopent-1-ene-1-carboxylic acid has a higher binding affinity to GABA-AT as compared to CPP-115 (K₁ values of 1 and CPP-115 have been found to be 9.7 μM and 59 μM, respectively), and (S)-3-amino-4-(difluoromethylenyl)cyclopent-1-ene-1-carboxylic acid inactivates GABA-AT at a greater rate than CPP-115 (k_{inact} values of (S)-3-amino-4-(difluoromethylenyl)cyclopent-1-ene-1-carboxylic acid and CPP-115 were 3.32 min⁻¹ and 2.05 mm⁻¹, respectively). See, U.S. Pat. No. 9,993,449. Overall, the efficiency constant for (S)-3-amino-4-(difluoromethylenyl)cyclopent-1-ene-1-carboxylic acid (k_{inact}/K₁=342 mM⁻¹min⁻¹) is 9.8 times larger than that for CPP-115 (k_{inact}/K₁=34.9 mM⁻¹min⁻¹); therefore, (S)-3-amino-4-(difluoromethylenyl)cyclopent-1- ene-1-carboxylic acid is 9.8 times more efficient as an inactivator of GABA-AT than CPP-115. Unlike vigabatrin, CPP-115 was reported not to inactivate or inhibit off-target enzymes, such as aspartate aminotransferase (Asp-AT) and alanine aminotransferase (Ala-AT), which could have contributed to its larger margin of safety than vigabatrin. (S)-3-amino-4-(difluoromethylenyl)cyclopent-1-ene-1-carboxylic acid is a very weak reversible inhibitor of both Asp-AT and Ala-AT with an IC₅₀>4 mM. CPP-115 is a moderate inactivator of OAT with a K₁ value of 0.116 mM and a k_{inact} value of 0.097 min⁻¹. (S)-3-amino-4-(difluoromethylenyl)cyclopent-1-ene-1-carboxylic acid is a potent inactivator of OAT with a K₁ value of 0.0033 mM and a K_{inact} value of 0.025 min⁻¹. By comparison of the K_{inact}/K₁ value of (S)-3-amino-4-(difluoromethylenyl)cyclopent-1-ene-1-carboxylic acid (7.6 mM⁻¹min⁻¹) with that of CPP-115 (0.84 mM⁻¹min⁻¹). (S)-3-amino-4-(difluoromethylenyl)cyclopent-1-ene-1-carboxylic acid is 9.0 times more efficient an inactivator of OAT than CPP-115, consistent with its higher efficiency as an inactivator of GABA-AT.

Without wishing to be bound by any particular theory, selective inhibition of GABA-AT by (S)-3-amino-4-(difluoromethylenyl)cyclopent-1-ene-1-carboxylic acid or a pharmaceutically acceptable salt thereof, raises GABA concentrations where GABA is normally present. By increasing GABA concentrations GABA_{A}-Rs are activated which inhibits the damaging inflammatory activity of lymphocytes and macrophages in the pancreas while also reducing production of inflammatory cytokines, thus inhibiting the disease process. In addition, diminished levels of GABA and an altered GABAergic signaling system contribute to the pathogenesis of diabetes and prediabetes. Administration of (S)-3-amino-4-(difluoromethylenyl)cyclopent-1-ene-1-carboxylic acid or a pharmaceutically acceptable salt thereof increases GABA concentrations resulting in increased insulin secretion, inhibition of immune response, improved β-cells survival, facilitation of changing of α cells into β-cells in the pancreas, and modulation of the GABAergic signaling system, thus treating and improving symptoms of type 1 diabetes, type 2 diabetes and prediabetes.

In embodiments, the terms “effective amount” or “therapeutically effective amount” may be used interchangeably and refer to an amount of a compound, material, composition, medicament, or other material that is effective to achieve reduction, elimination or prophylaxis of diabetes or prediabetes with a minimum of side effects normally associated with hypoglycemic agents or other GABA-AT inhibitors.

In embodiments, an “effective amount” herein can range from about 0.01 mg to about 750 mg of (S)-3-amino-4-(difluoromethylenyl)cyclopent-1-ene-1-carboxylic acid or a pharmaceutically acceptable salt thereof given from one to four times or more a day. For example, a pharmaceutical composition including an effective amount of (S)-3-amino-4-(difluoromethylenyl)cyclopent-1-ene-1-carboxylic acid or a pharmaceutically acceptable salt thereof may contain from about 0.01 mg to about 0.1 mg, about 0.1 mg to about 1 mg, 1 mg to about 5 mg, about 5 mg to about 10 mg, about 10 mg to about 15 mg, about 15 mg to about 20 mg, about 20 mg to about 25 mg, about 25 mg to about 30 mg, about 30 mg to about 35 mg, about 35 mg to about 40 mg, about 40 mg to about 45 mg, about 45 mg to about 50 mg, about 50 mg to about 55 mg, about 55 mg to about 60 mg, about 60 mg to about 65 mg, about 65 mg to about 70 mg, about 70 mg to about 75 mg, about 75 mg to about 80 mg, about 80 mg to about 85 mg, about 85 mg to about 90 mg, about 90 mg to about 95 mg, about 95 mg to about 100 mg, about 100 mg to about 105 mg, about 105 mg to about 110 mg, about 110 mg to about 115 mg, about 115 mg to about 120 mg, about 120 mg to about 125 mg, about 125 mg to about 130 mg, about 130 mg to about 135 mg, about 135 mg to about 140 mg, about 140 mg to about 145 mg, about 145 mg to about 150, about 150 mg to about 155 mg, about 155 mg to about 160 mg, about 160 mg to about 165 mg, about 165 mg to about 170 mg, about 170 mg to about 175 mg, about 175 mg to about 180 mg, about 180 mg to about 185 mg, about 185 mg to about 190 mg, about 190 mg to about 195 mg, about 195 mg to about 200 mg, about 200 mg to about 205 mg, about 205 mg to about 210 mg, about 210 mg to about 215 mg, about 215 mg to about 220 mg, about 220 mg to about 225 mg, about 225 mg to about 230 mg, about 230 mg to about 235 mg, about 235 mg to about 240 mg, about 240 mg to about 245 mg, about 245 mg to about 250, about 250 mg to about 255 mg, about 255 mg to about 260 mg, about 260 mg to about 265 mg, about 265 mg to about 270 mg, about 270 mg to about 275 mg, about 275 mg to about 280 mg, about 280 mg to about 285 mg, about 285 mg to about 290 mg, about 290 mg to about 295 mg, about 295 mg to about 300 mg, about 300 mg to about 305 mg, about 305 mg to about 310 mg, about 310 mg to about 315 mg, about 315 mg to about 320 mg, about 320 mg to about 325 mg, about 325 mg to about 330 mg, 330 mg to about 335 mg, about 335 mg to about 340 mg, about 340 mg to about 345 mg, about 345 mg to about 350, about 350 mg to about 355 mg, about 355 mg to about 360 mg, about 360 mg to about 365 mg, about 365 mg to about 370 mg, about 370 mg to about 375 mg, about 375 mg to about 380 mg, about 380 mg to about 385 mg, about 385 mg to about 390 mg, about 390 mg to about 395 mg, about 395 mg to about 400 mg, about 400 mg to about 405 mg, about 405 mg to about 410 mg, about 410 mg to about 415 mg, about 415 mg to about 420 mg, about 420 mg to about 425 mg, about 425 mg to about 430 mg, about 430 mg to about 435 mg, about 435 mg to about 440 mg, about 440 mg to about 445 mg, about 445 mg to about 450, about 450 mg to about 455 mg, about 455 mg to about 460 mg, about 460 mg to about 465 mg, about 465 mg to about 470 mg, about 470 mg to about 475 mg, about 475 mg to about 480 mg, about 480 mg to about 485 mg, about 485 mg to about 490 mg, about 490 mg to about 495 mg, or about 495 mg to about 500 mg of (S)-3-amino-4-(difluoromethylenyl)cyclopent-1-ene-1-carboxylic acid or a pharmaceutically acceptable salt thereof.

In embodiments a pharmaceutical composition containing an effective amount of (S)-3-amino-4-(difluoromethylenyl)cyclopent-1-ene-1-carboxylic acid or a pharmaceutically acceptable salt thereof includes 0.01 mg, 0.1 mg, 0.5 mg, 1 mg, 2 mg, 3 mg, 4 mg, 5 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, 90 mg, 91 mg, 92mg, 93 mg, 94 mg, 95 mg, 96 mg, 97 mg, 98 mg, 99 mg, 100 mg, 101 mg, 102 mg, 103 mg, 104 mg, 105 mg, 106 mg, 107 mg, 108 mg, 109 mg, 110 mg, 111 mg, 112 mg, 113 mg, 114 mg, 115 mg, 116 mg, 117 mg, 118 mg, 119 mg, 120 mg, 121 mg, 122 mg, 123 mg, 124 mg, 125 mg, 126 mg, 127 mg, 128 mg, 129 mg, 130 mg, 131 mg, 132 mg, 133 mg, 134 mg, 135 mg, 136 mg, 137 mg, 138 mg, 139 mg, 140 mg, 141 mg, 142 mg, 143 mg, 144 mg, 145 mg, 146 mg, 147 mg, 148 mg, 149 mg, 150 mg, 151 mg, 152 mg, 153 mg, 154 mg, 155 mg, 156 mg, 157 mg, 158 mg, 159 mg, 160 mg, 161 mg, 162 mg, 163 mg, 164 mg, 165 mg, 166 mg, 167 mg, 168 mg, 169 mg, 170 mg, 171 mg, 172 mg, 173 mg, 174 mg, 175 mg, 176 mg, 177 mg, 178 mg, 179 mg, 180 mg, 181 mg, 182 mg, 183 mg, 184 mg, 185 mg, 186 mg, 187 mg, 188 mg, 189 mg, 190 mg, 191 mg, 192 mg, 193 mg, 194 mg, 195 mg, 196 mg, 197 mg, 198 mg, 199 mg, 200 mg, 201 mg, 202 mg, 203 mg, 204 mg, 205 mg, 206 mg, 207 mg, 208 mg, 209 mg, 210 mg, 211 mg, 212 mg, 213 mg, 214 mg, 215 mg, 216 mg, 217 mg, 218 mg, 219 mg, 220 mg, 221 mg, 222 mg, 223 mg, 224 mg, 225 mg, 226 mg, 227 mg, 228 mg, 229 mg, 230 mg, 231 mg, 232 mg, 233 mg, 234 mg, 235 mg, 236 mg, 237 mg, 238 mg, 239 mg, 240 mg, 241 mg, 242 mg, 243 mg, 244 mg, 245 mg, 246 mg, 247 mg, 248 mg, 249 mg, 250 mg, 251 mg, 252 mg, 253 mg, 254 mg, 255 mg, 256 mg, 257 mg, 258 mg, 259 mg, 260 mg, 261 mg, 262 mg, 263 mg, 264 mg, 265 mg, 266 mg, 267 mg, 268 mg, 269 mg, 270 mg, 271 mg, 272 mg, 273 mg, 274 mg, 275 mg, 276 mg, 277 mg, 278 mg, 279 mg, 280 mg, 281 mg, 282 mg, 283 mg, 284 mg, 285 mg, 286 mg, 287 mg, 288 mg, 289 mg, 290 mg, 291 mg, 292 mg, 293 mg, 294 mg, 295 mg, 296 mg, 297 mg, 298 mg, 299 mg, 300 mg, 301 mg, 302 mg, 303 mg, 304 mg, 305 mg, 306 mg, 307 mg 308 mg, 309 mg, 310 mg, 311 mg, 312 mg, 313 mg, 314 mg, 315 mg, 316 mg, 317 mg, 318 mg, 319 mg, 320 mg, 321 mg, 322 mg, 323 mg, 324 mg, 325 mg, 326 mg, 327 mg, 328 mg, 329 mg, 230 mg, 331 mg, 332 mg, 333 mg, 334 mg, 335 mg, 336 mg, 337 mg, 338 mg, 339 mg, 340 mg, 241 mg, 342 mg, 343 mg, 344 mg, 345 mg, 346 mg, 347 mg, 348 mg, 349 mg, 350 mg, 351 mg, 352 mg, 353 mg, 354 mg, 355 mg, 356 mg, 357 mg, 358 mg, 359 mg, 360 mg, 361 mg, 362 mg, 363 mg, 364 mg, 365 mg, 366 mg, 367 mg, 368 mg, 369 mg, 370 mg, 371 mg, 372 mg, 373 mg, 374 mg, 375 mg, 376 mg, 377 mg, 378 mg, 379 mg, 380 mg, 381 mg, 382 mg, 383 mg, 384 mg, 385 mg, 386 mg, 387 mg, 388 mg, 389 mg, 390 mg, 391 mg, 392 mg, 393 mg, 394 mg, 395 mg, 396 mg, 397 mg, 398 mg, 399 mg, 400 mg, 401 mg, 402 mg, 403 mg, 404 mg, 405 mg, 406 mg, 407 mg, 408 mg, 409 mg, 410 mg, 411 mg, 412 mg, 413 mg, 414 mg, 415 mg, 416 mg, 417 mg, 418 mg, 419 mg, 420 mg, 421 mg, 422 mg, 423 mg, 424 mg, 425 mg, 426 mg, 427 mg, 428 mg, 429 mg, 430 mg, 431 mg, 432 mg, 433 mg, 434 mg, 435 mg, 436 mg, 237 mg, 438 mg, 439 mg, 440 mg, 441 mg, 442 mg, 443 mg, 444 mg, 445 mg, 446 mg, 447 mg, 448 mg, 449 mg, 450 mg, 451 mg, 452 mg, 453 mg, 454 mg, 455 mg, 456 mg, 457 mg, 458 mg, 459 mg, 460 mg, 461 mg, 462 mg, 463 mg, 464 mg, 465 mg, 466 mg, 467 mg, 468 mg, 469 mg, 470 mg, 471 mg, 472 mg, 473 mg, 474 mg, 475 mg, 476 mg, 477 mg, 478 mg, 479 mg, 480 mg, 481 mg, 482 mg, 483 mg, 484 mg, 485 mg, 486 mg, 487 mg, 488 mg, 489 mg, 490 mg, 491 mg, 492 mg, 493 mg, 494 mg, 495 mg, 496 mg, 497 mg, 498 mg, 499 mg, or 500 mg, of (S)-3-amino-4-(difluoromethylenyl)cyclopent-1-ene-1-carboxylic acid or a pharmaceutically acceptable salt thereof.

In embodiments, (S)-3-amino-4-(difluoromethylenyl)cyclopent-1-ene-1-carboxylic acid or a pharmaceutically acceptable salt thereof is administered to a subject at from about 0.01 mg/per day to about 750 mg/per day. For example, in embodiments, (S)-3-amino-4-(difluoromethylenyl)cyclopent-1-ene-1-carboxylic acid or a pharmaceutically acceptable salt thereof is administered to a subject at about 0.01 mg/per day, about 0.1 mg/per day, about 0.5 mg/per day about 1 mg/per day, about 5 mg/per day, about 10 mg/per day, about 15 mg/per day, about 20 mg/per day, about 25 mg/per day, about 30 mg/per day, about 35 mg/per day, about 40 mg/per day, about 45 mg/per day, about 50 mg/per day, about 60 mg/per day, about 65 mg/per day, about 70 mg/per day, about 75 mg/per day, about 80 mg/per day, about 85 mg/per day, about 90 mg/per day, about 95 mg/per day, about 100 mg/per day, about 105 mg/per day, about 110 mg/per day, about 115 mg/per day, about 120 mg/per day, about 125 mg/per day, about 130 mg/per day, about 135 mg/per day, about 140 mg/per day, about 145 mg/per day, about 150 mg/per day, about 155 mg/per day, about 160 mg/per day, about 165 mg/per day, about 170 mg/per day, about 175 mg/per day, about 180 mg/per day, about 185 mg/per day, about 190 mg/per day, about 195 mg/per day, about 200 mg/per day, about 205 mg/per day, about 210 mg/per day, about 215 mg/per day, about 220 mg/per day, about 225 mg/per day, about 230 mg/per day, about 235 mg/per day, about 240 mg/per day, about 245 mg/per day, about 250 mg/per day, about 255 mg/per day, about 260 mg/per day, about 265 mg/per day, about 270 mg/per day, about 275 mg/per day, about 280 mg/per day, about 285 mg/per day, about 290 mg/per day, about 295 mg/per day, about 300 mg/per day, about 305 mg/per day, about 310 mg/per day, about 315 mg/per day, about 320 mg/per day, about 325 mg/per day, about 330 mg/per day, about 335 mg/per day, about 340 mg/per day, about 345 mg/per day, about 350 mg/per day, about 355 mg/per day, about 360 mg/per day, about 365 mg/per day, about 370 mg/per day, about 375 mg/per day, about 380 mg/per day, about 385 mg/per day, about 390 mg/per day, about 395 mg/per day, about 400 mg/per day, about 405 mg/per day, about 410 mg/per day, about 415 mg/per day, about 420 mg/per day, about 425 mg/per day, about 430 mg/per day, about 435 mg/per day, about 440 mg/per day, about 445 mg/per day, about 450 mg/per day, about 455 mg/per day, about 460 mg/per day, about 465 mg/per day, about 470 mg/per day, about 475 mg/per day, about 480 mg/per day, about 485 mg/per day, about 490 mg/per day, about 495 mg/per day, about 500 mg/per day, about 505 mg/per day, about 510 mg/per day, about 515 mg/per day, about 520 mg/per day, about 525 mg/per day, about 530 mg/per day, about 535 mg/per day, about 540 mg/per day, about 545 mg/per day, about 550 mg/per day, about 555 mg/per day, about 560 mg/per day, about 565 mg/per day, about 570 mg/per day, about 575 mg/per day, about 580 mg/per day, about 585 mg/per day, about 590 mg/per day, about 595 mg/per day, about 600 mg/per day, about 605 mg/per day, about 610 mg/per day, about 615 mg/per day, about 620 mg/per day, about 625 mg/per day, about 630 mg/per day, about 635 mg/per day, about 640 mg/per day, about 645 mg/per day, about 650 mg/per day, about 655 mg/per day, about 660 mg/per day, about 665 mg/per day, about 670 mg/per day, about 675 mg/per day, about 680 mg/per day, about 685 mg/per day, about 690 mg/per day, about 695 mg/per day, about 700 mg/per day, about 705 mg/per day, about 710 mg/per day, about 715 mg/per day, about 720 mg/per day, about 725 mg/per day, about 730 mg/per day, about 735 mg/per day, about 740 mg/per day, about 745 mg/per day, or about 750 mg/per day, in one, two, three, four or more doses. In embodiments, the subject may be started at a low dose and the dosage is escalated over time.

In embodiments, (S)-3-amino-4-(difluoromethylenyl)cyclopent-1-ene-1-carboxylic acid or a pharmaceutically acceptable salt thereof is administered to a subject diagnosed with diabetes or prediabetes via a pharmaceutical composition. Pharmaceutical compositions herein encompass dosage forms. Dosage forms herein encompass unit doses. In embodiments, as discussed below, various dosage forms including conventional formulations and modified release formulations can be administered one, two, three, four or more times daily. In embodiments, (S)-3-amino-4-(difluoromethylenyl)cyclopent-1-ene-1-carboxylic acid or a pharmaceutically acceptable salt thereof is administered to a subject once or twice a day, (e.g., morning and/or evening). In embodiments, (S)-3-amino-4-(difluoromethylenyl)cyclopent-1-ene-1-carboxylic acid or a pharmaceutically acceptable salt thereof is administered to a subject three times a day, (e.g., morning, afternoon and at bedtime, or every 8 hours). In embodiments, (S)-3-amino-4-(difluoromethylenyl)cyclopent-1-ene-1-carboxylic acid or a pharmaceutically acceptable salt thereof is administered to a subject four times a day, (e.g., morning, afternoon, evening and at bedtime, or every 6 hours). In embodiments, (S)-3-amino-4-(difluoromethylenyl)cyclopent-1-ene-1-carboxylic acid or a pharmaceutically acceptable salt thereof is administered to a subject via continuous infusion. In embodiments, (S)-3-amino-4-(difluoromethylenyl)cyclopent-1-ene-1-carboxylic acid or a pharmaceutically acceptable salt thereof is administered to a subject at the start of a diabetes or prediabetes symptom episode, whenever that may occur. Any suitable route of administration may be utilized, e.g., oral, rectal, nasal, pulmonary, vaginal, sublingual, transdermal, intravenous, intraarterial, epidural, intramuscular, intraperitoneal and subcutaneous routes. Suitable dosage forms include tablets, capsules, oral liquids, powders, acrosols, transdermal modalities such as topical liquids, patches, creams and ointments, parenteral formulations and suppositories. In embodiments, (S)-3-amino-4-(difluoromethylenyl)cyclopent-1-ene-1-carboxylic acid or a pharmaceutically acceptable salt thereof is used to manufacture a medicament for treatment of diabetes or prediabetes.

In embodiments, methods of treating diabetes or prediabetes are provided which include administering to a subject in need thereof a pharmaceutical composition including (S)-3-amino-4-(difluoromethylenyl)cyclopent-1-ene-1-carboxylic acid or a pharmaceutically acceptable salt thereof wherein the composition provides improvement in symptoms of diabetes or prediabetes for more than 1 hour after administration to the subject. In embodiments, methods of treating diabetes or prediabetes are provided which include administering to a subject in need thereof a pharmaceutical composition including (S)-3-amino-4-(difluoromethylenyl)cyclopent-1-ene-1-carboxylic acid or a pharmaceutically acceptable salt thereof wherein the composition provides improvement in symptoms of diabetes or prediabetes for more than 2 hours after administration to the subject. In embodiments, methods of treating diabetes or prediabetes are provided which include administering to a subject in need thereof a pharmaceutical composition including (S)-3-amino-4-(difluoromethylenyl)cyclopent-1-ene-1-carboxylic acid or a pharmaceutically acceptable salt thereof wherein the composition provides improvement in symptoms of diabetes or prediabetes for more than 3 hours after administration to the subject. In embodiments, methods of treating diabetes or prediabetes are provided which include administering to a subject in need thereof a pharmaceutical composition including (S)-3-amino-4-(difluoromethylenyl)cyclopent-1-ene-1-carboxylic acid or a pharmaceutically acceptable salt thereof wherein the composition provides improvement in symptoms of diabetes or prediabetes for more than 4 hours after administration to the subject. In embodiments, methods of treating diabetes or prediabetes are provided which include administering to a subject in need thereof a pharmaceutical composition including (S)-3-amino-4-(difluoromethylenyl)cyclopent-1-ene-1-carboxylic acid or a pharmaceutically acceptable salt thereof wherein the composition provides improvement in symptoms of diabetes or prediabetes for more than 6 hours after administration to the subject. In embodiments, methods of treating diabetes or prediabetes are provided which include administering to a subject in need thereof a pharmaceutical composition including (S)-3-amino-4-(difluoromethylenyl)cyclopent-1-ene-1-carboxylic acid or a pharmaceutically acceptable salt thereof wherein the composition provides improvement in symptoms of diabetes or prediabetes for more than 8, 10, 12, 14, 16, 18, 20, 22 or 24 hours after administration to the subject. In embodiments, the pharmaceutical compositions provide improvement of next day functioning of the subject diagnosed with diabetes or prediabetes. For example, the pharmaceutical compositions may provide improvement in symptoms of diabetes or prediabetes for more than about, e.g., 2 hours, 4 hours, 6 hours, 8 hours, 10 hours, 12 hours, 14 hours, 16 hours, 18 hours, 20 hours, 22 hours or 24 hours after administration and waking from a night of sleep.

In embodiments, as mentioned previously, pharmaceutical compositions herein may be provided with conventional release or modified release profiles. Pharmaceutical compositions may be prepared using a pharmaceutically acceptable “carrier” composed of materials that are considered safe and effective. The “carrier” includes all components present in the pharmaceutical formulation other than the active ingredient or ingredients. The term “carrier” includes, but is not limited to, diluents, binders, lubricants, disintegrants, fillers, and coating compositions. Those with skill in the art are familiar with such pharmaceutical carriers and methods of compounding pharmaceutical compositions using such carriers.

In embodiments, pharmaceutical compositions herein are modified release dosage forms which provide modified release profiles. Modified release profiles may exhibit immediate release, delayed release, or extended release profiles. Conventional (or unmodified) release oral dosage forms such as tablets, capsules, suppositories, syrups, solutions and suspensions typically release medications into the mouth, stomach or intestines as the tablet, capsule shell or suppository dissolves, or, in the case of syrups, solutions and suspensions, when they are swallowed. The pattern of drug release from modified release (MR) dosage forms is deliberately changed from that of a conventional dosage form to achieve a desired therapeutic objective and/or better patient compliance. Types of MR drug products include orally disintegrating dosage forms (ODDFs) which provide immediate release, extended release dosage forms, delayed release dosage forms (e.g., enteric coated), and pulsatile release dosage forms.

An ODDF is a solid dosage form containing a medicinal substance or active ingredient which disintegrates rapidly, usually within a matter of seconds when placed upon the tongue. The disintegration time for ODDFs generally range from one or two seconds to about a minute. ODDFs are designed to disintegrate or dissolve rapidly on contact with saliva. This mode of administration can be beneficial to people who may have problems swallowing tablets whether it be from physical infirmity or psychiatric in nature. Subjects with diabetes or prediabetes may exhibit such behavior. ODDF's can provide rapid delivery of medication to the blood stream through mucosa resulting in a rapid onset of action. Examples of ODDFs include orally disintegrating tablets, capsules and rapidly dissolving films and wafers.

Extended release dosage forms (ERDFs) have extended release profiles and are those that allow a reduction in dosing frequency as compared to that presented by a conventional dosage form, e.g., a solution or unmodified release dosage form. ERDFs provide a sustained duration of action of a drug. Suitable formulations which provide extended release profiles are well-known in the art. For example, coated slow release beads or granules (“beads” and “granules” are used interchangeably herein) in which (S)-3-amino-4-(difluoromethylenyl)cyclopent-1-ene-1-carboxylic acid or a pharmaceutically acceptable salt thereof is applied to beads, e.g., confectioners nonpareil beads, and then coated with conventional release retarding materials such as waxes, enteric coatings and the like. In embodiments, beads can be formed in which (S)-3-amino-4-(difluoromethylenyl)cyclopent-1-ene-1-carboxylic acid or a pharmaceutically acceptable salt thereof is mixed with a material to provide a mass from which the drug leaches out. In embodiments, the beads may be engineered to provide different rates of release by varying characteristics of the coating or mass, e.g., thickness, porosity, using different materials, etc. Beads having different rates of release may be combined into a single dosage form to provide variable or continuous release. The beads can be contained in capsules or compressed into tablets.

In embodiments, modified dosage forms herein incorporate delayed release dosage forms having delayed release profiles. Delayed release dosage forms can include delayed release tablets or delayed release capsules. A delayed release tablet is a solid dosage form which releases a drug (or drugs) such as (S)-3-amino-4-(difluoromethylenyl)cyclopent-1-ene-1-carboxylic acid or a pharmaceutically acceptable salt thereof at a time other than promptly after administration. A delayed release capsule is a solid dosage form in which the drug is enclosed within either a hard or soft soluble container made from a suitable form of gelatin, and which releases a drug (or drugs) at a time other than promptly after administration. For example, enteric-coated tablets, capsules, particles and beads are well-known examples of delayed release dosage forms. Enteric coated tablets, capsules and particles and beads pass through the stomach and release the drug in the intestine. In embodiments, a delayed release tablet is a solid dosage form containing a conglomerate of medicinal particles that releases a drug (or drugs) at a time other than promptly after administration. In embodiments, the conglomerate of medicinal particles is covered with a coating which delays release of the drug. In embodiments, a delayed release capsule is a solid dosage form containing a conglomerate of medicinal particles that releases a drug (or drugs) at a time other than promptly after administration. In embodiments, the conglomerate of medicinal particles is covered with a coating which delays release of the drug.

Delayed release dosage forms are known to those skilled in the art. For example, coated delayed release beads or granules in which (S)-3-amino-4-(difluoromethylenyl)cyclopent-1-ene-1-carboxylic acid or a pharmaceutically acceptable salt thereof is applied to beads, e.g., confectioners nonpareil beads, and then coated with conventional release delaying materials such as waxes, enteric coatings and the like. In embodiments, beads can be formed in which (S)-3-amino-4-(difluoromethylenyl)cyclopent-1-ene-1-carboxylic acid or a pharmaceutically acceptable salt thereof is mixed with a material to provide a mass from which the drug leaches out. In embodiments, the beads may be engineered to provide different rates of release by varying characteristics of the coating or mass, e.g., thickness, porosity, using different materials, etc. In embodiments, enteric coated granules of (S)-3-amino-4-(difluoromethylenyl)cyclopent-1-ene-1-carboxylic acid or a pharmaceutically acceptable salt thereof can be contained in an enterically coated capsule or tablet which releases the granules in the small intestine. In embodiments, the granules have a coating which remains intact until the coated granules reach at least the ileum and thereafter provide a delayed release of the drug in the colon. Suitable enteric coating materials are well known in the art, e.g., Eudragit® coatings such methacrylic acid and methyl methacrylate polymers and others. The granules can be contained in capsules or compressed into tablets.

In embodiments, (S)-3-amino-4-(difluoromethylenyl)cyclopent-1-ene-1-carboxylic acid or a pharmaceutically acceptable salt thereof is incorporated into porous inert carriers that provide delayed release profiles. In embodiments, the porous inert carriers incorporate channels or passages from which the drug diffuses into surrounding fluids. In embodiments, (S)-3-amino-4-(difluoromethylenyl)cyclopent-1-ene-1-carboxylic acid or a pharmaceutically acceptable salt thereof is incorporated into an ion-exchange resin to provide a delayed release profile. Delayed action may result from a predetermined rate of release of the drug from the resin when the drug-resin complex contacts gastrointestinal fluids and the ionic constituents dissolved therein. In embodiments, membranes are utilized to control rate of release from drug containing reservoirs. In embodiments, liquid preparations may also be utilized to provide a delayed release profile. For example, a liquid preparation consisting of solid particles dispersed throughout a liquid phase in which the particles are not soluble. The suspension is formulated to allow at least a reduction in dosing frequency as compared to that drug presented as a conventional dosage form (e.g., as a solution or a prompt drug-releasing, conventional solid dosage form). For example, a suspension of ion-exchange resin constituents or microbeads.

In embodiments, pharmaceutical compositions described herein are suitable for parenteral administration, including, e.g., intramuscular (i.m.), intravenous (i.v.), subcutaneous (s.c.), intraperitoneal (i.p.), epidural, or intrathecal (i.t.). Parenteral compositions should be sterile for administration by injection, infusion or implantation into the body and may be packaged in either single-dose or multi-dose containers. In embodiments, liquid pharmaceutical compositions for parenteral administration to a subject include an active substance, e.g., (S)-3-amino-4-(difluoromethylenyl)cyclopent-1-ene-1-carboxylic acid or a pharmaceutically acceptable salt thereof in any of the respective amounts described above. In embodiments, the pharmaceutical compositions for parenteral administration are formulated as a total volume of about, e.g., 10 ml, 20 ml, 25 ml, 50 ml, 100 ml, 200 ml, 250 ml, or 500 ml. In embodiments, the compositions are contained in a bag, a glass vial, a plastic vial, or a bottle.

Pharmaceutical compositions for parenteral administration provided herein may include one or more excipients, e.g., solvents, solubility enhancers, suspending agents, buffering agents, isotonicity agents, stabilizers or antimicrobial preservatives. When used, the excipients of the parenteral compositions will not adversely affect the stability, bioavailability, safety, and/or efficacy of (S)-3-amino-4-(difluoromethylenyl)cyclopent-1-ene-1-carboxylic acid or a pharmaceutically acceptable salt thereof used in the composition. Thus, parenteral compositions are provided wherein there is no incompatibility between any of the components of the dosage form.

In embodiments, parenteral compositions (S)-3-amino-4-difluoromethylenyl)cyclopent-1-ene-1-carboxylic acid or a pharmaceutically acceptable salt thereof include a stabilizing amount of at least one excipient. For example, excipients may be selected from the group consisting of buffering agents, solubilizing agents, tonicity agents, antioxidants, chelating agents, antimicrobial agents, and preservative. One skilled in the art will appreciate that an excipient may have more than one function and be classified in one or more defined group.

In embodiments, parenteral compositions (S)-3-amino-4-difluoromethylenyl)cyclopent-1-ene-1-carboxylic acid or a pharmaceutically acceptable salt thereof and an excipient wherein the excipient is present at a weight percent (w/v) of less than about, e.g., 10%, 5%, 2.5%, 1%, or 0.5%. In embodiments, the excipient is present at a weight percent between about, e.g., 1.0% to 10%, 10% to 25%, 15% to 35%, 0.5% to 5%, 0.001% to 1%, 0.01% to 1%, 0.1% to 1%, or 0.5% to 1%. In embodiments, the excipient is present at a weight percent between about, e.g., 0.001% to 1%, 0.01% to 1%, 1.0% to 5%, 10% to 15%, or 1% to 15%.

In embodiments, parenteral compositions of an active substance, e.g., (S)-3-amino-4-(difluoromethylenyl)cyclopent-1-ene-1-carboxylic acid or a pharmaceutically acceptable salt thereof are provided, wherein the pH of the composition is between about 4.0 to about 8.0. In embodiments, the pH of the compositions is between, e.g., about 5.0 to about 8.0, about 6.0 to about 8.0, about 6.5 to about 8.0. In embodiments, the pH of the compositions is between, e.g., about 6.5 to about 7.5, about 7.0 to about 7.8, about 7.2 to about 7.8, or about 7.3 to about 7.6. In embodiments, the pH of the aqueous solution is, e.g., about 6.8, about 7.0, about 7.2, about 7.4, about 7.6, about 7.7, about 7.8, about 8.0, about 8.2, about 8.4, or about 8.6.

It should be understood that the dosage amounts of (S)-3-amino-4-(difluoromethylenyl)cyclopent-1-ene-1-carboxylic acid, or a pharmaceutically acceptable salt thereof that are provided herein are applicable to all the dosage forms described herein including conventional dosage forms, modified dosage forms, as well as the parenteral formulations described herein. Those skilled in the art will determine appropriate amounts depending on criteria such as dosage form, route of administration, subject tolerance, efficacy, therapeutic goal and therapeutic benefit, among other pharmaceutically acceptable criteria.

Clinical efficacy of treatment can be monitored using any method known in the art. Measurable parameters to monitor efficacy will depend on the condition being treated. For monitoring the status or improvement of diabetes or prediabetes, both subjective parameters (e.g., patient reporting) and objective parameters (e.g., fasting blood glucose levels, HbA1c, 2-hour oral glucose tolerance test (OGTT) result level, and random plasma glucose level) can be used.

Unless defined otherwise, all technical and scientific terms used herein have the same meanings as commonly understood by one of skill in the art to which the disclosure herein belongs.

The term “about” or “approximately” as used herein means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, up to 10%, up to 5%, and/or up to 1% of a given value.

“Improvement” refers to the treatment of diabetes or prediabetes including all symptoms that are normally associated with diabetes or prediabetes.

“Improvement in next day functioning” or “wherein there is improvement in next day functioning” refers to improvement after waking from an overnight sleep period wherein the beneficial effect of administration of (S)-3-amino-4-(difluoromethylenyl)cyclopent-1-ene-1-carboxylic acid or a pharmaceutically acceptable salt thereof applies to symptoms diabetes or prediabetes and is discernable, either subjectively by a subject or objectively by an observer, for a period of time, e.g., 2 hours, 3 hours, 4 hours, 5 hours, 6 hours, 12 hours, 24 hours, etc. after waking.

“Treating”, “treatment” or “treat” can refer to the following: reducing, improving, relieving, ameliorating, mitigating, inhibiting, reversing and/or alleviating diabetes or prediabetes in a subject, or delaying the appearance of symptoms of diabetes or prediabetes (prophylaxis) in a subject. In embodiments, “treating”, “treat” or “treatment” may refer to preventing the appearance of clinical symptoms of a disease or condition in a subject that may be afflicted with or predisposed to the disease or condition but does not yet experience or display clinical or subclinical symptoms of the disease or condition. “Treating”, “treat” or “treatment” also refers to inhibiting or relieving diabetes or prediabetes, e.g., causing regression of diabetes or prediabetes or at least one of its clinical or subclinical symptoms. The benefit to a subject to be treated may be statistically significant, mathematically significant, or at least perceptible to the subject and/or the physician. Nonetheless, prophylactic (preventive) and therapeutic (curative) treatment are two separate embodiments of the disclosure herein.

“Pharmaceutically acceptable” refers to molecular entities and compositions that are “generally regarded as safe”, e.g., that are physiologically tolerable and do not typically produce an allergic or similar untoward reaction, such as gastric upset and the like, when administered to a human. In embodiments, this term refers to molecular entities and compositions approved by a regulatory agency of the federal or a state government, as the GRAS list under section 204(s) and 409 of the Federal Food, Drug and Cosmetic Act, that is subject to premarket review and approval by the FDA or similar lists, the U.S. Pharmacopeia or another generally recognized pharmacopeia for use in animals, and more particularly in humans.

“Co-administered with”, “administered in combination with”, “a combination of” or “administered along with” may be used interchangeably and mean that two or more agents are administered in the course of therapy. The agents may be administered together at the same time or separately in spaced apart intervals. The agents may be administered in a single dosage form or in separate dosage forms.

“Subject in need thereof” includes individuals that have been diagnosed with diabetes or prediabetes. Individuals include mammals. The methods and compositions including (S)-3-amino-4-(difluoromethylenyl)cyclopent-1-ene-1-carboxylic acid or a pharmaceutically acceptable salt thereof may be provided to any individual including, e.g., wherein the subject is a neonate, infant, a pediatric subject (6 months to 12 years), an adolescent subject (age 12-18 years) or an adult (over 18 years). Subjects include mammals. “Patient” and “subject” are used interchangeably herein.

The term “pharmaceutically acceptable salt”, as used herein, refers to derivatives of the compounds defined herein, wherein the parent compound is modified by making acid or base salts thereof. Examples of pharmaceutically acceptable salts include but are not limited to mineral or organic acid salts of basic residues such as amines; and alkali or organic salts of acidic residues such as carboxylic acids. The pharmaceutically acceptable salts include the conventional non-toxic salts or the quaternary ammonium salts of the parent compound formed, for example, from non-toxic inorganic or organic acids. Such conventional non-toxic salts include but are not limited to those derived from inorganic acids such as hydrochloric, hydrobromic, sulfuric, sulfamic, phosphoric, and nitric acids; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, tolunesulfonic, naphthalenesulfonic, methanesulfonic, ethane disulfonic, oxalic, and isethionic salts. The pharmaceutically acceptable salts can be synthesized from the parent compound, which contains a basic or acidic moiety, by conventional chemical methods.

It should be understood that the examples and embodiments provided herein are exemplary examples and embodiments. Those skilled in the art will envision various modifications of the examples and embodiments that are consistent with the scope of the disclosure herein. Such modifications are intended to be encompassed by the claims.

What is claimed is:

1. A method of treating diabetes comprising administering an effective amount of (S)-3-amino-4-(difluoromethylenyl)cyclopent-1-ene-1-carboxylic acid or a pharmaceutically acceptable salt thereof to a subject in need thereof.

2. The method of treating diabetes according to claim 1, wherein the diabetes is type 2 diabetes.

3. The method of treating diabetes according to claim 1, wherein the diabetes is type 1 diabetes.

4. The method of treating diabetes according to claim 1, wherein the (S)-3-amino-4-(difluoromethylenyl)cyclopent-1-ene-1-carboxylic acid or a pharmaceutically acceptable salt thereof is administered to a subject in need thereof in an amount effective to lower one or more of HbA1c level, fasting plasma glucose level, 2-hour oral glucose tolerance test (OGTT) result level, and random plasma glucose level.

5. The method of treating diabetes according to claim 4, wherein the HbA1c level is lowered by an amount greater than 0.5%.

6. The method of treating diabetes according to claim 4, wherein the HbA1c level is lowered by an amount greater than 1.0%.

7. The method of treating diabetes according to claim 1, wherein the (S)-3-amino-4-(difluoromethylenyl)cyclopent-1-ene-1-carboxylic acid or a pharmaceutically acceptable salt thereof is administered in an amount ranging from about 0.01 mg to about 750 mg.

8. The method of treating diabetes according to claim 1, wherein the (S)-3-amino-4-(difluoromethylenyl)cyclopent-1-ene-1-carboxylic acid or a pharmaceutically acceptable salt thereof is administered in combination with one or more hypoglycemic agents selected from the group consisting of a biguanide, a dipeptidyl peptidase-4 (DPP-4) inhibitor, a sulphonylurea, a thiazolidinedione, a glinide, an alpha-glucosidase blocker, an glucagon-like peptide-1 receptor agonist, insulin and an insulin analog.

9. A method of treating prediabetes comprising administering an effective amount of (S)-3-amino-4-(difluoromethylenyl)cyclopent-1-ene-1-carboxylic acid or a pharmaceutically acceptable salt thereof to a subject in need thereof.

10. The method of treating prediabetes according to claim 9, wherein the (S)-3-amino-4-(difluoromethylenyl)cyclopent-1-ene-1-carboxylic acid or a pharmaceutically acceptable salt thereof is administered to a subject in need thereof in an amount effective to lower one or more of HbA1c level, fasting plasma glucose level, 2-hour oral glucose tolerance test (OGTT) result level, and random plasma glucose level.

11. The method of treating prediabetes according to claim 10, wherein the HbA1c level is lowered by an amount greater than 0.5%.

12. The method of treating prediabetes according to claim 10, wherein the HbA1c level is lowered by an amount greater than 1.0%.

13. The method of treating prediabetes according to claim 9, wherein the (S)-3-amino-4-(difluoromethylenyl)cyclopent-1-ene-1-carboxylic acid or a pharmaceutically acceptable salt thereof is administered in an amount ranging from about 0.01 mg to about 750 mg.

14. The method of treating prediabetes according to claim 9, wherein the (S)-3-amino-4-(difluoromethylenyl)cyclopent-1-ene-1-carboxylic acid or a pharmaceutically acceptable salt thereof is administered in combination with one or more hypoglycemic agents selected from the group consisting of a biguanide, a dipeptidyl peptidase-4 (DPP-4) inhibitor, a sulphonylurea, a thiazolidinedione, a glinide, an alpha-glucosidase blocker, an glucagon-like peptide-1 receptor agonist, insulin and an insulin analog.

15. A method of treating diabetes and sleep apnea comprising administering (S)-3-amino-4-(difluoromethylenyl)cyclopent-1-ene-1-carboxylic acid or a pharmaceutically acceptable salt thereof to a subject diagnosed with the diabetes and sleep apnea in an amount effective to reduce episodes of sleep apnea.

16. The method of treating diabetes and sleep apnea according to claim 15, wherein the diabetes disease is type 2 diabetes.

17. The method of treating diabetes and sleep apnea according to claim 15, wherein the diabetes is type I diabetes.

18. The method of treating diabetes and sleep apnea according to claim 15, wherein the (S)-3-amino-4-(difluoromethylenyl)cyclopent-1-ene-1-carboxylic acid or a pharmaceutically acceptable salt thereof is administered in an amount ranging from about 0.01 mg to about 750 mg.

19. The method of treating diabetes and sleep apnea according to claim 15, wherein the (S)-3-amino-4-(difluoromethylenyl)cyclopent-1-ene-1-carboxylic acid or a pharmaceutically acceptable salt thereof is administered in combination with one or more hypoglycemic agents selected from the group consisting of a biguanide, a dipeptidyl peptidase-4 (DPP-4) inhibitor, a sulphonylurea, a thiazolidinedione, a glinide, an alpha-glucosidase blocker, an glucagon-like peptide-1 receptor agonist, insulin and an insulin analog.

20. The method of treating diabetes and sleep apnea according to claim 15, wherein the administering is effective to lower one or more of HbA1c level, fasting plasma glucose level, 2-hour oral glucose tolerance test (OGTT) result level, and random plasma glucose level in the subject.

21. The method of treating diabetes and sleep apnea according to claim 20, wherein the HbA1c level is lowered by an amount greater than 0.5%.

22. The method of treating diabetes and sleep apnea according to claim 20, wherein the HbA1c level is lowered by an amount greater than 1%.
